(19) United States
(12) Patent Application Publication
(10) Pub. No.: US 2010/0137716 A1
(43) Pub. Date: Jun. 3, 2010

(54) MULTI-FUNCTIONAL ULTRASOUND IMAGING SYSTEM

(76) Inventors: Zhenyu Liu, Wuxi (CN); Huiren Chen, Wuxi (CN)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/625,278

(22) Filed: Nov. 24, 2009

(30) Foreign Application Priority Data
Nov. 28, 2008 (CN) 200810182374.4

(51) Int. Cl. A61B 8/14

(52) U.S. Cl. 600/447

(57) ABSTRACT

A multi-functional ultrasound imaging system includes a scan imaging unit having a transmitting unit, a receiving unit, and a probe that is one of a linear array probe and a convex array probe, wherein the probe has a certain deflection angle. The system also includes a control unit configured to control said scan imaging unit to realize one of normal linear scan and additional sector scan, normal convex scan and additional sector scan. The system also includes an image processing unit configured to process image signals generated by said scan imaging unit and to convert the image signals into image data corresponding to an ultrasound image, and a display unit configured to generate the ultrasound image corresponding to the image data.

### CROSS REFERENCE TO RELATED APPLICATIONS

This application claims the benefit of Chinese Patent Application No. 200810182374.4 filed Nov. 28, 2008, which is hereby incorporated by reference in its entirety.

### BACKGROUND OF THE INVENTION

The embodiments described herein relate to an ultrasound imaging system, and particularly relates to a multi-functional scan technique in a medical ultrasound imaging system.

Traditional ultrasound scanner support several types of probes, such as convex array probe, micro-convex array probe, linear array probe and sector probe. Each type probe can be used for special clinical application. Usually, a convex array probe is preferably used for abdomen scanning, a linear array probe is preferable used for superficial parts (such as mammary gland and thyroid gland, etc.) scanning, a sector probe is preferably used for small acoustic window (such as Cardiac). Except for some special-purpose scanners, most ultrasound scanners cover multi-range applications by using different probes, but this requires an operator to change probes to perform scanning to different parts of body during operation. For example, the console type ultrasound scanner always has two or more probe ports and supports electric-mechanical switch, but compact type ultrasound scanner usually has only one probe port. If an operator needs to move to scan another part, he has to disconnect the currently used probe and connect the probe to be used in the next step. Moreover, using more than one probes will increase disinfection steps in a surgery, and this wastes time which is very precious in emergency room. Besides, more cables and parts are not easy of usage, especially in emergency room, surgical room or at ambulance.

### BRIEF DESCRIPTION OF THE INVENTION

In some embodiments, a multi-functional ultrasound imaging system is provided for use with in a medical imaging system, which uses one probe to realize scanning of more than two types.

One aspect provides a multi-functional ultrasound imaging system, comprising: a scan imaging unit including an transmitting unit, a receiving unit and a linear array probe or convex array probe having a certain deflection angle; a control unit for controlling the scan imaging unit to realize normal linear scan and the additional sector scan or to realize the normal convex scan and the additional sector scan; an image processing unit for processing image signals generated by the scan imaging unit to convert them into image data corresponding to an ultrasound image; a display unit for generating an ultrasound image corresponding to said image data.

In some embodiments, the sector scan can be normal sector scan or additional sector scan.

In some embodiments, a linear array probe and convex array probe having a deflection angle of more than 25° is designed or selected such that the linear array probe and convex array probe can achieve a particular effect in sector scan, and the preferable deflection angle is between 30° and 45°.

In some embodiments, the multi-functional ultrasound imaging system further comprises an operating unit inputting a command to the control unit by an operator to perform the normal scan or the additional sector scan.

In some embodiments, the operating unit is arranged in the control unit; said operating unit can be a button, a voice controlled switch, a touch panel, or a key on a keyboard.

In another aspect, a multi-functional ultrasound imaging system provides an additional sector scan or additional extended sector scan on a normal linear array probe or a convex array probe so as to realize providing two fields of view (FOV) of an ultrasound image on a linear array probe or a convex array probe. Although the additional sector scan or extended sector scan generated by the multi-functional ultrasound imaging system according to the present invention cannot achieve the precise effect of a single sector scan probe, but it saves the precious time especially in an emergent case in the emergency room, e.g. reducing the number of times of changing probes by a doctor when there is a need to scan the abdomen and cardiac parts, moreover, it provides better image quality as compared with using the current one linear array probe or convex array probe to scan two types of body parts.

### BRIEF DESCRIPTION OF THE DRAWINGS

FIGS. 1A, 1B, and 1C illustrate three types of linear array probes that generate different scan sequences in the prior art respectively;

FIGS. 2A and 2B illustrate two types of convex array probes that generate different scan sequences in the prior art respectively;

FIG. 3 is a block diagram of the multi-functional ultrasound imaging system according to the present invention;

FIG. 4 illustrates the processing process of a scan sequence transmitted by the transmitting unit of the ultrasound imaging system via a probe;

FIG. 5 illustrates the processing process of receiving from a probe a scan sequence by the receiving unit of the ultrasound imaging system;

FIG. 6 illustrates a simple process of generating a linear shape image by the multi-functional linear ultrasound imaging system according to the present invention;

FIG. 7 illustrates a simple process of generating a sector image by the multi-functional linear ultrasound imaging system according to the present invention;

FIG. 8A is an illustration of generating sector scan by the multi-functional linear ultrasound imaging system according to the present invention;

FIG. 8B is an illustration of generating sector scan by the multi-functional convex ultrasound imaging system according to the present invention;

FIG. 9A is an illustration of generating an extended sector scan sequence by the multi-functional linear ultrasound imaging system according to the present invention.

FIG. 9B is an illustration of generating an extended sector scan sequence by the multi-functional convex ultrasound imaging system according to the present invention.

### DETAILED DESCRIPTION OF THE INVENTION

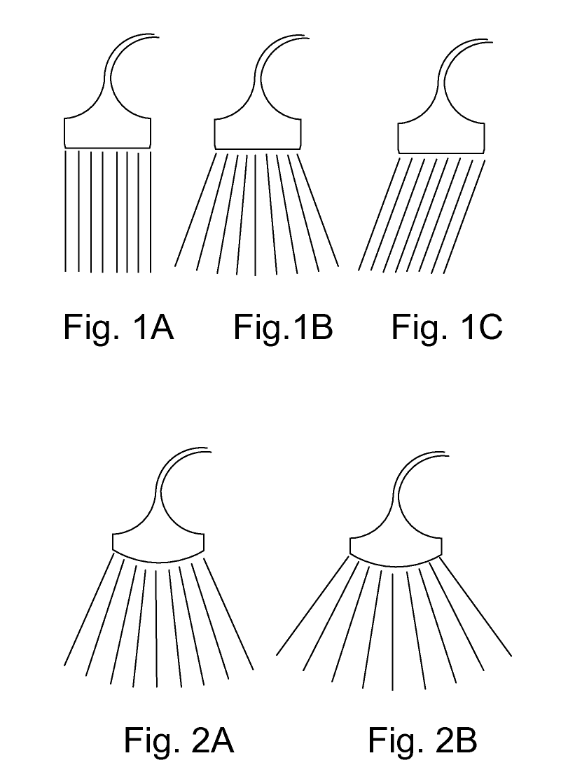

For a usual linear array probe, as shown in FIGS. 1A, 1B, and 1C respectively, different linear array probes can have different directivity according to the requirement in designing, i.e., the deflection angle of other beams with respect to the central beam of the linear array probe. The linear array probe shown in FIGS. 1A and 1C is an ideal linear array probe which has a deflection angle of 0; the linear array probe shown in FIG. 1B has a certain deflection angle. In actual application, the deflection angle is made as small as possible, normally within 25° to ensure the linear scan effect of a linear array probe. The effect of sector scan using such a linear array probe is bad. In order to make a linear array probe realize appropriate sector scan so as to reduce the times of switching probes and the steps of disinfection in emergency room, surgical room or at ambulance, the he present invention newly designs or selects the current linear array probe having a deflection angle of more than 25°, e.g. between 30° and 60°, preferable between 35° to 45°, such that the linear array probe can perform both linear scan and sector scan.

For normal convex array probes, as shown in FIGS. 2A and 2B, different convex array probes can have different directivity according to the requirement in designing, i.e., the deflection angle of other beams with respect to the central beam of the convex array probe. The convex array probe shown in FIG. 2A has a smaller deflection angle than the convex array probe shown in FIG. 2B. The present invention may select a convex array probe having an appropriate deflection angle for realizing sector scan on a convex array probe.

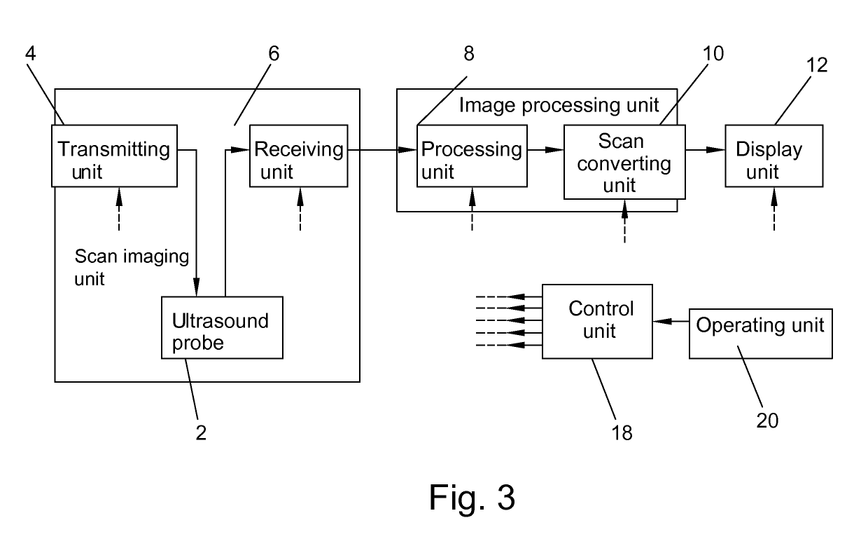

FIG. 3 is a block diagram of the multi-functional ultrasound imaging system according to the present invention. As shown in FIG. 3, the multi-functional ultrasound imaging system according to the present invention comprises a scan imaging unit including an transmitting unit 4, a receiving unit 6 and an ultrasound probe 2, said ultrasound probe 2 is a linear array probe or a convex array probe having a certain deflection angle; a control unit 18 for controlling the scan imaging unit to perform normal linear scan and additional sector scan, or to perform normal convex scan and additional sector scan; an image processing unit for processing an image signal generated by the scan imaging unit so as to convert it into image data corresponding to an ultrasound image; a display unit 12 for displaying an ultrasound image corresponding to said image data.

In the ultrasound imaging system according to the present invention, the image processing unit comprises a processing unit 8 and a scan converting unit 10, the image processing unit may further comprises a buffer unit according to requirement. Since the basic function and operation of these units are well known to those skilled in the art, no detailed introduction is made herein.

Besides, the multi-functional ultrasound imaging system shown in FIG. 3 may further comprise an operating unit 20 for an operator to input a command to the control unit 18 to make the control unit 18 perform desired control. Said operating unit 20 can be arranged within the control unit 18 or independent from the control unit 18. For example, said operating unit can be a key on the keyboard operated by the operator, or a button, a voice switch or a touch panel.

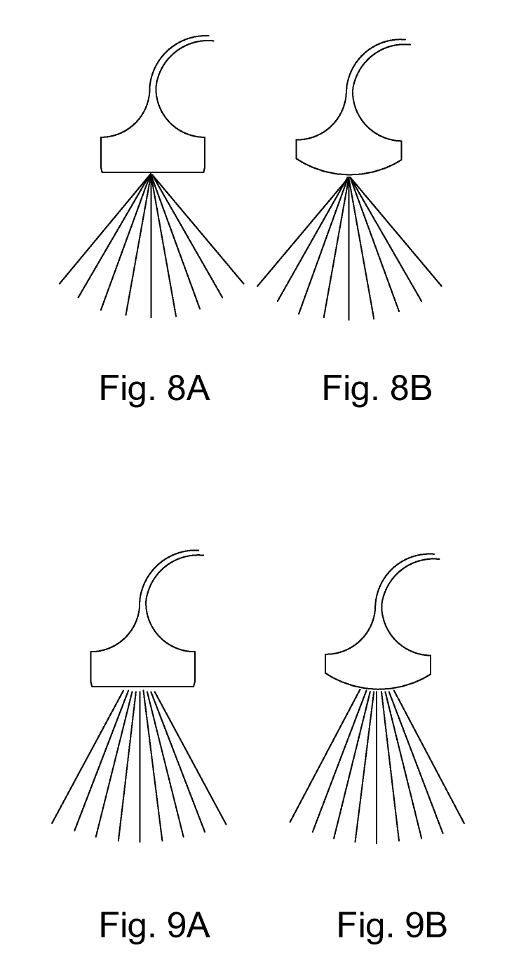

In the multi-functional ultrasound imaging system shown in FIG. 3, said sector scan can be a normal sector scan or extended sector scan. FIG. 9A is an illustration of the additional extended sector scan generated by the multi-functional linear ultrasound imaging system; FIG. 9B is an illustration of the additional extended sector scan generated by the multi-functional convex ultrasound imaging system.

In the multi-functional ultrasound imaging system shown in FIG. 3, in order to make a linear array probe that normally generates a linear scan sequence or a convex array probe that normally generates a convex scan sequence generate additional sector scan or extended sector scan, a current linear array probe or convex array probe having a big deflection angle as required is selected or a linear array probe or convex array probe having a big deflection angle as required is newly designed such that it not only supports normal linear scan or normal convex scan, but also supports additional sector scan or additional extended sector scan. The deflection angle of said linear array probe and convex array probe is larger than 25°, preferably, between 30° and 45°. Besides, a digital beamformer in the receiving unit 6 and the scan converting unit 10 as well as display unit 12 are also designed to support said two types of scan.

Moreover, it should be pointed out that the combination of the units shown in FIG. 3 is only one embodiment. The present invention can have other combinations in accordance with the actual requirement or arrange the units as separate independent components. In a word, any combination, arrangement and transformation of these components that can realize the purpose of the present invention falls within the scope of protection of the present invention.

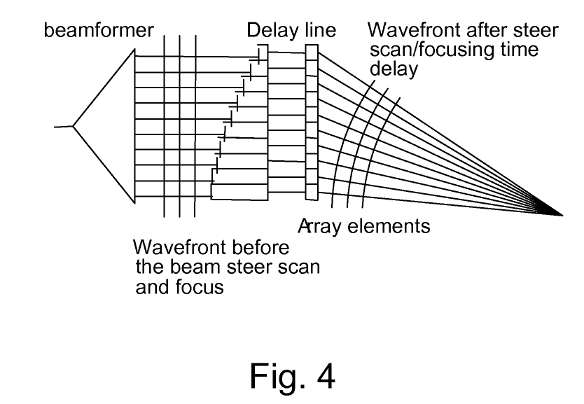

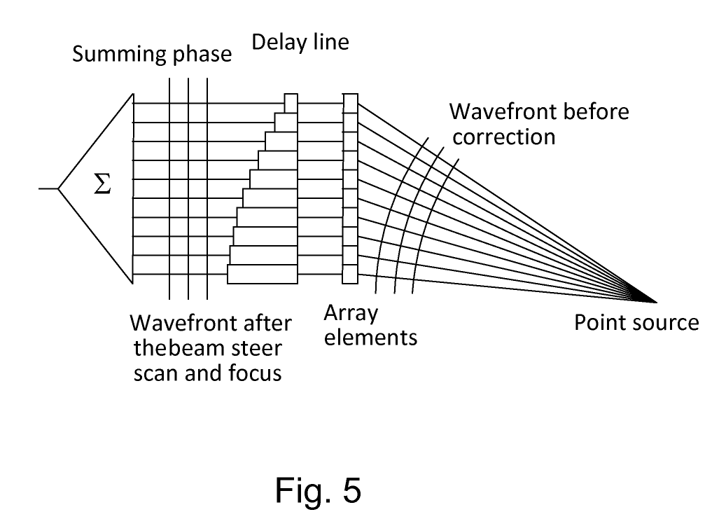

FIG. 4 illustrates the basic principle of the transmitting portion of the linear ultrasound imaging system; FIG. 5 illustrates the basic principle of the receiving portion of the linear ultrasound imaging system. The ultrasound emission and reception use the superposition and interference principle of sound fields. Normally, as shown in FIG. 4, several groups of ultrasound transducers in a linear array probe are arranged in a linear array, the control unit 18 controls the beamformer in the transmitting unit 4 to transmit ultrasound waves, the delay of the ultrasound waves are adjusted through the corresponding delay line before the ultrasound waves reaches the transducer array, the delayed ultrasound waves continuously motivate the groups of transducers sequentially such that the ultrasound waves transmitted by the array elements in the groups of transducers superimpose and synthesize in the space to form the desired focusing and/or deflected scan beam. After scanning the part to be examined, said scan beam generates an echo signal which is received by the transducer array, as shown in FIG. 5. The transducer array transmits the received echo signal to the receiving unit 6, in which delay of the corresponding echo signal is adjusted through a delay line to form parallel ultrasound beams, and the echo signal is strengthened through synthesization. When a group of transducer has completely received the echo, the next adjacent group of transducers starts to work. The echo signals received in the receiving unit 6 are transmitted to the processing unit 8 and the scan converting unit 10 and are converted image data of the ultrasound image after processing and scan conversion, the ultrasound image is then displayed on the display unit 12. Wherein the delay lines shown in FIGS. 4 and 5 can be analog or digital, and the digital delay line is widely adopted for being flexible, precise and easy to be controlled by a computer.

The multi-functional ultrasound imaging system according to the present invention transmits and receives pulses along the sector scan direction by using a new scan sequence controlling beamformer; then the scan converting unit and display unit present the echo information as an image based on the geometrical arrangement of the sector.

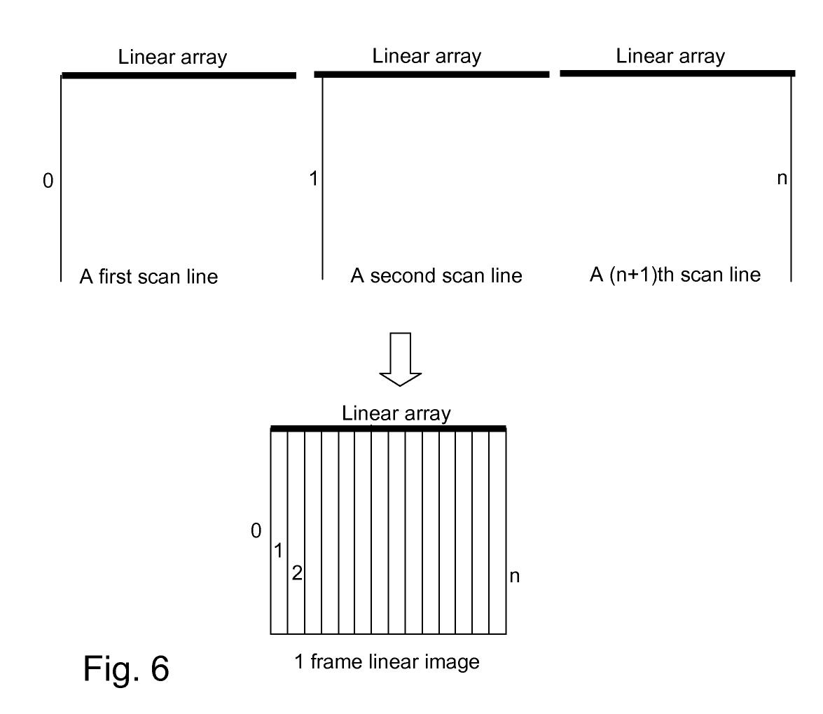

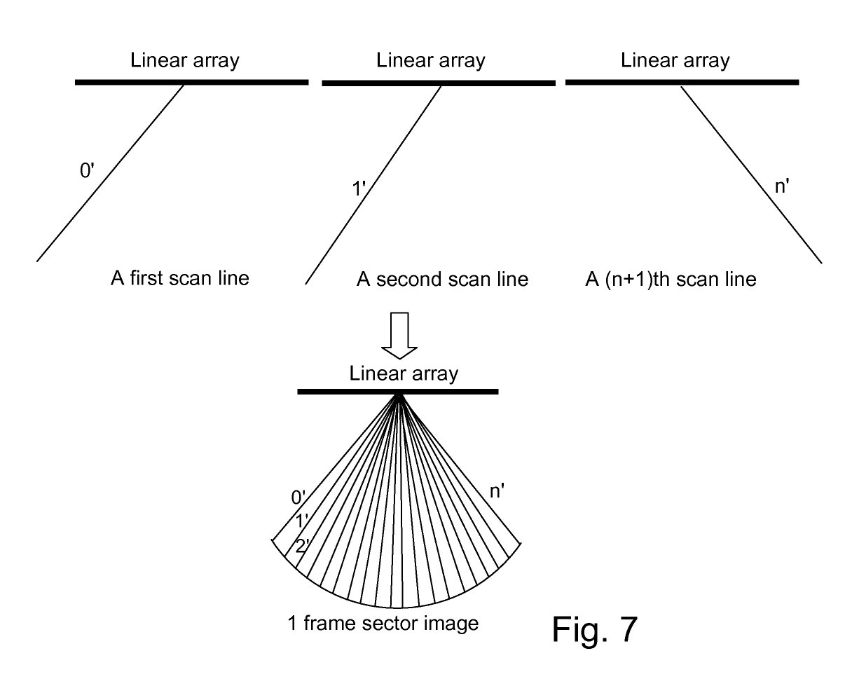

Embodiments of the invention are described taking a multi-functional linear ultrasound imaging system as an example in the following text: FIG. 8A is an illustration of the additional sector scan generated by the multi-functional linear ultrasound imaging system according to the present invention. The multi-functional linear ultrasound imaging system has a structure as shown in FIG. 3, which can perform normal linear scan and additional sector scan. When the multi-functional linear ultrasound imaging system is desired to generate a normal linear image, an operator operates the operating unit 20 to make the control unit 18 control the transmitting unit 4 to transmit normal linear sequences, as shown in FIG. 6, a first scan line 0, a second scan line 1, . . . a (n+1)th scan line n that are parallel with one another, and control the receiving unit 6 to receive the parallel linear scan line sequences returned from the transducer array of the linear array probe 2, which are then processed by the processing unit 8 and scan converted in the scan converting unit 10, a linear image is then displayed on the display unit 12. When the multi-functional linear array probe is desired to generate a sector image, the operator operates the operating unit 20 such that the control unit 18 controls the transmitting unit 4 to transmit sector sequences, as shown in FIG. 7, a first scan line 0′, a second scan line 1′, . . . , a (n+1)th scan line n′ having a certain deflection angle, namely, generating a sector scan line sequence having a certain deflection angle, and controls the receiving unit 6 to receive the sector scan line sequences returned from the transducer array of the linear array probe 2, which are then processed by the processing unit 8 and scan converted in the scan converting unit 10, a sector image is then displayed on the display unit 12.

From FIGS. 6 and 7 it can be seen that the multi-functional linear ultrasound imaging system according to said embodiment of the present invention can generate scan of two different FOV, one is a relatively narrow normal linear scan (shown in FIG. 6), which can be used in detection of superficial parts; the other is a relatively broad sector scan (shown in FIG. 7), which can be used in sector scan of small acoustic windows (such as Cardiac).

Similarly, a multi-functional convex ultrasound imaging system can be provided in accordance with the present invention, which can generate normal convex scan and additional sector scan. FIG. 8A shows an illustration of a sector scan array generated by the multi-functional convex ultrasound imaging system according to the present invention.

Based on said concepts of the present invention, sector scan and convex scan can also be realized on a linear array probe; a linear scan and sector scan can be realized on a convex array probe. That is, the present invention is not limited to using one probe to realize two types of scan.

More than two types of scan can be realized as required.

The above description is only a specific mode of carrying out the present invention. It should be pointed out that those skilled in the art can make improvement, revision and transformation without deviating from the spirit of the present invention. These improvement, revision and transformation shall all be deemed as fall within the scope of protection of the present application.

1. A multi-functional ultrasound imaging system comprising:
a scan imaging unit comprising:
a transmitting unit;
a receiving unit; and
a probe comprising one of a linear array probe and a convex array probe, said probe having a certain deflection angle;
a control unit configured to control said scan imaging unit to realize one of normal linear scan and additional sector scan, normal convex scan and additional sector scan;
an image processing unit configured to process image signals generated by said scan imaging unit and to convert the image signals into image data corresponding to an ultrasound image; and
a display unit configured to generate the ultrasound image corresponding to the image data.

2. The multi-functional ultrasound imaging system according to claim 1, wherein the additional sector scan is one of normal sector scan and extended sector scan.

3. The multi-functional ultrasound imaging system according to claim 1, wherein the deflection angle of said probe is larger than 25°.

4. The multi-functional ultrasound imaging system according to claim 3, wherein the deflection angle of said probe is between 30° and 45°.

5. The multi-functional ultrasound imaging system according to claim 1, further comprising:
an operating unit configured to receive a command input by an operator to said control unit to perform one of normal scan and additional sector scan.

6. The multi-functional ultrasound imaging system according to claim 5, wherein said control unit comprises said operating unit.

7. The multi-functional ultrasound imaging system according to claim 5, wherein said operating unit comprises at least one of a button, a voice control switch, a touch panel and a key on a keyboard.

8. An ultrasound imaging method comprising:
performing one of normal linear scan and additional sector scan, and normal convex scan and additional sector scan using a scan imaging unit;
processing image signals generated by the scan imaging unit using an imaging processing unit;
converting the image signals into image data corresponding to an ultrasound image using the imaging processing unit; and
generating the ultrasound image corresponding to the image data using a display unit.

9. The ultrasound imaging method according to claim 8, wherein the additional sector scan is one of normal sector scan and extended sector scan.

10. The ultrasound imaging method according to claim 8, wherein the deflection angle of a probe within the scan imaging unit is larger than 25°.

11. The ultrasound imaging method according to claim 10, wherein the deflection angle of the probe is between 30° and 45°.

12. The ultrasound imaging method according to claim 8, further comprising:
receiving a command input from an operator via an operating unit to perform one of normal scan and additional sector scan.

13. The ultrasound imaging method according to claim 12, wherein the operating unit is arranged within a control unit.

14. The ultrasound imaging method according to claim 12, wherein receiving a command comprises receiving a command via at least one of a button, a voice control switch, a touch panel, and a key on a keyboard.

15. An ultrasound imaging system comprising:
a scan imaging unit comprising:
an transmitting unit;
a receiving unit; and
a probe comprising one of a linear array probe and a convex array probe, said probe having a certain deflection angle;
a control unit configured to control said scan imaging unit to realize one of normal linear scan and additional sector scan, normal convex scan and additional sector scan;
an operating unit configured to receive a command input by an operator to said control unit to perform one of normal scan and additional sector scan;
an image processing unit configured to process image signals generated by said scan imaging unit and to convert the image signals into image data corresponding to an ultrasound image; and
a display unit configured to generate the ultrasound image corresponding to the image data.

16. The ultrasound imaging system according to claim 15, wherein the additional sector scan is one of normal sector scan and extended sector scan.

17. The ultrasound imaging system according to claim 15, wherein the deflection angle of said probe is larger than 25°.

18. The multi-functional ultrasound imaging system according to claim 17, wherein the deflection angle of said probe is between 30° and 45°.

19. The ultrasound imaging system according to claim 15, wherein said control unit comprises said operating unit.

20. The ultrasound imaging system according to claim 15, wherein said operating unit comprises at least one of a button, a voice control switch, a touch panel, and a key on a keyboard.
